(19) United States
(12) Patent Application Publication
(10) Pub. No.: US 2009/0233372 A1
(43) Pub. Date: Sep. 17, 2009

(54) COMPOSITIONS, KITS AND METHODS FOR DETERMINING ETIOLOGY OF TRALI AND DETECTING PATIENTS AT RISK FOR THIS TRANSFUSION REACTION

(75) Inventor: Daniel Ambruso, Denver, CO (US)

Correspondence Address: PATTON BOGGS LLP, 8484 WESTPARK DRIVE, SUITE 900, MCLEAN, VA, 22102, US

(73) Assignee: Bonfils Blood Center, Denver, CO (US)

(21) Appl. No.: 12/401,109

(22) Filed: Mar. 10, 2009

Related U.S. Application Data
(60) Provisional application No. 61/064,517, filed on Mar. 10, 2008.

(51) Int. Cl. G01N 33/50

(52) U.S. Cl. 436/68

(57) ABSTRACT

The instant application is to compositions, kits and methods to determine if a person in need of a blood transfusion is at-risk for TRALI. The invention includes embodiments of methods for testing the priming activity of a blood component or serum or plasma from a patient sustaining TRALI or the priming status of neutrophils of a patient at risk for TRALI by exposing the neutrophils to samples or priming agents, and measuring the respiratory burst in response to an activating agent. The respiratory burst may then be compared to a pre-determined value to find if the patient has abnormally high respiratory burst or the plasma or serum samples have priming activity. The present invention also contemplates kits designed to measure respiratory burst, and compositions/reagents to be used in same.

### CROSS-REFERENCE TO RELATED APPLICATIONS

This application claims priority to U.S. Provisional Application No. 61/064,517, filed Mar. 10, 2008, the contents of which are incorporated herein in its entirety.

### BACKGROUND OF THE INVENTION

1. Field of the Invention

The present invention generally relates to use of compositions, kits and methods to determine if a person in need of a blood transfusion is at risk for Transfusion Related Acute Lung Injury (TRALI) or any related condition or if a patient has sustained TRALI as a result of biologic response modifiers in the transfused blood component.

2. Background

Transfusion Related Acute Lung Injury (TRALI) is a lung injury that is temporally related to a blood transfusion, and is one of the leading causes of transfusion-related fatalities in the United States. TRALI is best described as a clinical constellation of signs and symptoms including hypoxia, dyspnea, cyanosis, hypotension, fever and chills along with physical and radiographic findings of bilateral pulmonary infiltrates. Notably, TRALI is accompanied by damage to endothelial cells, an inflammatory response in the lung and thickening the alveolar wall, thus reducing the oxygen transport by the lung. The symptoms may begin during or within 1-2 hours of transfusion and usually are present by 6 hours after the infusion. The severity can range from mild to severe but is related to the extent of damage and degree of hypoxia. TRALI occurs in anywhere from 1 in every 1,000 to 1 in every 100,000 transfusions, depending upon the study. The severe pulmonary damage and hypoxia associated with hypo- or hypertension, fever and sometimes leukopenia may be fatal in a subset of patients (about 5-10% of reported cases), while those that survive usually recover and return to normal pulmonary function within 48-96 hours. TRALI now represents the most common cause of transfusion-related death.

Currently, TRALI is thought to be due to an uncontrolled host inflammatory response with neutrophil activation targeted predominantly at the pulmonary capillary beds. The pathophysiology of these reactions is dependent on the presence of both primed and activated neutrophils and endothelial cells and their interaction. The mechanisms of TRALI have been observed in models of acute lung injury. For instance, in male Long Evans rats, instillation of polyclonal rabbit IgG anti-bovine serum albumin (BSA), followed by the infusion of BSA, results in the deposition of immune complexes along alveolar walls, complement activation and the accumulation of large numbers of neutrophils recruited from the blood. Interstitial edema, intra-alveolar hemorrhage and fibrin deposition then occurs, resulting in major damage to the pulmonary vasculature and endothelial cells. While not a direct model of TRALI, this process provides insights into the process of neutrophil activation and recruitment.

Currently, there are two “models” of TRALI hypothesized. In the first model, leukocyte antibodies in donors activate recipient neutrophils in pulmonary capillaries and cause capillary leak and pulmonary damage.

In the second model, the TRALI is caused by two events. The first event is linked to the patient's underlying condition at the time of the transfusion, such as sepsis or coronary bypass surgery that leads to stimulation and activation of the vascular endothelium and priming of neutrophils. This results in sequestering neutrophils in the pulmonary vasculature. The second event is the transfusion of blood containing biologically active substances; for example, lipids or cytokines, which prime and activate neutrophils, leading to lung damage and capillary leak. The adhesion and activation of neutrophils causes endothelial damage in alveolar capillaries and further transmigration of inflammation. Thus, there is a breakdown in the normal vascular anatomy, with massive fluid leakage and inflammation into the alveoli.

Supporting this latter hypothesis is the finding that noxious factors in stored blood plasma causes lung injury in a canine model (Geelhoed and Bennett, Am Surg 1975;41:661-682). In humans, leukocyte antibodies have been detected in only 3.6% of TRALI reactions in the University of Alberta study, and neutrophil priming activity was greater in implicated units than in control units (Silliman, et al, Blood 2003;101:454-462 and Silliman, et al, Transfusion 1997;37:719-726). Lipids accumulated during storage are present in cellular blood components, and these lipids have neutrophil priming activity. Researchers have found that priming activity and lipids were increased in samples taken from patients at the time of the TRALI reaction. Furthermore, lipids detected in the plasma of stored red cells and platelets caused TRALI in an ex vivo rat lung model.

The two-event model is also pertinent for the antibody-mediated TRALI; antibody can be the second event in a primed patient, and thus the two models are not mutually exclusive. Thus, in both models, the neutrophils may be sequestered and activated on the epithelial surface by the first and second event, respectively.

Neutrophils participate in host defense against bacterial, fungal and viral infections by phagocytosing and killing the invading microbes. The killing depends largely on activation of the respiratory burst provoking the formation of reactive oxygen metabolites consisting of superoxide anions, hydroxyl radicals, hydrogen peroxide and singlet oxygen. The respiratory burst can be initiated by a variety of stimuli including, but not limited to, tumor necrosis factor a, the chemotactic peptide fMLP, and 4-phorbol-12-β-myristate-13-acetate (PMA). Most of these interact with target cell constituents or receptors which activate a cascade of biochemical reactions ultimately causing assembly and activation of the NADPH oxidase resulting in consumption of oxygen and production of superoxide anion. Other reactive oxygen species (ROS) such as hydrogen peroxide, hypochlorous acid, hydroxyl radical and singlet oxygen. Further, the reactive oxygen metabolites, such as superoxide anion, may interact with chromophores producing a specific chemical product such as reduced cytochrome c.

Currently, no prospective strategies are in place to prevent TRALI, other than the deferral of donors previously implicated in severe cases. Proposals for clinical interventions have included the deferral of all multiparous females, the segregation of female plasma to fractionation with the use of only male plasma in clinical practice, and the possibility of testing donor plasma for specific antibodies. The United Kingdom has chosen to segregate female plasma from fractionation based on limited data showing the plasma, and specifically, plasma from females who have antibodies is most frequently associated with TRALI in their hemovigilance studies. Currently, this restriction has been proposed by the American Association of Blood Banks and has been adopted by most blood centers and transfusion services in the United States to reduce the risk of TRALI. Testing of apheresis platelet donors for antibodies as a deferral scheme is being evaluated.

As such, there is an acute need for a method of preventing TRALI or documenting the existence of etiologic agents to confirm occurrence in blood transfusion patients or predicating when an occurrence may arise. In addition, there is a need to generally predict the severity of a potential response. Further, there is a need for an easy to use kit to detect patients at risk for TRALI.

### BRIEF SUMMARY OF THE INVENTION

One or more of these and other needs are met through the instantly claimed use of compositions, kits and methods to determine if transfused blood contains etiologic agents associated with TRALI, if the patients who sustain TRALI have etiologic agents in their blood, or if a person in need of a blood transfusion is at-risk for TRALI. Thus, in its broadest sense, the invention provides compositions, kits and methods to determine if a person who has received blood and has an adverse reaction has TRALI or if a person in need of a blood transfusion is at risk for TRALI and the level of that risk.

One embodiment of the invention is to a method for testing the ability of the blood component or blood from the patient to prime neutrophils or the priming state of the neutrophils of a patient in need of a blood transfusion, the method comprising exposing the neutrophils to a neutrophil priming agent and, subsequently, a standard activating agent and measuring the release of respiratory burst and its products by the neutrophils, where the measured enhanced release of respiratory burst after priming compared to the activating agent alone is an index of the likelihood of a TRALI occurrence. In another embodiment, the chemicals released by the neutrophils and measured by the method are one or more reactive oxygen species (ROS).

Another embodiment of the invention is to a method for testing the priming activity of the blood component, the blood from the patient, or the priming state of the neutrophils of a patient in need of a blood transfusion, which comprises exposing neutrophils to a neutrophil priming agent and, subsequently, to an activating agent and measuring the ROS released by the neutrophils. In one embodiment, the ROS are measured by use of a cytochrome c assay. Superoxide anions reduce cytochrome c; thus, increased neutrophil respiratory burst results in further reduction of cytochrome c. One embodiment of the present invention is an assay for use in determining what chemicals or methods can lower the sensitivity of priming activity by plasma or serum from blood components or patient who has sustained TRALI or of the primed neutrophils in a patient at risk for a TRALI occurrence.

In one embodiment, the priming state of the neutrophils is measured by cytochrome c level determination, where the cytochrome c reduction is determined by use of an enzymatic or immunochemical method. In another embodiment, the cytochrome c level is determined by electrophoresis. In yet another embodiment, the cytochrome c level is determined by chromatography.

While the most common way to measure superoxide anion is enzymatically by measuring superoxide dismutase inhibitable cytochrome c reduction, other techniques may be used such as reduction of the dye nitro blue tetrazolium. Techniques using chemiluminescent probes and measuring chemiluminescence, fluorescent probes such as dihydrorhodamine, or specific electron traps and specific adducts by electron spin resonance (ESR) can also be used.

Another embodiment is to a kit for testing priming activity of blood samples from the blood component associated with the reaction or blood from the patient at the time of the reaction or the priming state of the neutrophils of a patient in need of a blood transfusion. The kit comprises at least one neutrophil priming agent and compounds for measuring chemicals released by neutrophils. Such a kit can be used to determine the etiology of a TRALI reaction or priming state of the neutrophils or as an assay for blocking the priming state of classes of neutrophils.

Another embodiment is to compositions used in the above methods and/or kits for testing the priming of neutrophils.

Other objects, features, and advantages of the present invention will become apparent with reference to the drawings and detailed description that follow.

### BRIEF DESCRIPTION OF FIGURES

FIG. 1A is a flow chart in accordance with some embodiments of the invention, demonstrating a method of determining the presence of etiologic agents or the risk for TRALI.

FIG. 1B is a flow chart in accordance with some embodiments of the invention, demonstrating a method of determining the presence of etiologic agents or the risk for TRALI.

FIG. 2 is a flow chart in accordance with some embodiments of the invention, demonstrating a method of determining the presence of etiologic agents or the risk for TRALI.

FIG. 3 is a chart summarizing superoxide anion production by neutrophils as cytochrome c reduction.

FIG. 4 is a chart evaluating neutrophil priming with PAF at Days 0 and 7 of storage.

FIG. 5 demonstrates the results of a priming assay using fluorescent probe for hydrogen peroxide.

FIG. 6 also demonstrates the results of a priming assay using fluorescent probe for hydrogen peroxide.

FIG. 7 is a chart showing chemiluminescence in response to fMLP alone and PAF priming of the fMLP response.

FIG. 8 is a chart demonstrating the effect of priming the fMLP response.

### DETAILED DESCRIPTION OF THE PREFERRED EMBODIMENT

In the following detailed description of the preferred embodiments, reference is made to the accompanying drawings that form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

One embodiment is to a method for testing for priming activity of blood components associated with TRALI, the priming activity of blood from a patient sustaining TRALI, or the priming state of the neutrophils of a patient in need of a blood transfusion, the method comprising exposing the neutrophils to the test plasma or agent, then an activating agent and measuring chemicals released by the neutrophils and using the determined measurement as an index of likelihood of TRALI occurrence.

### Exemplary Embodiments of the Invention

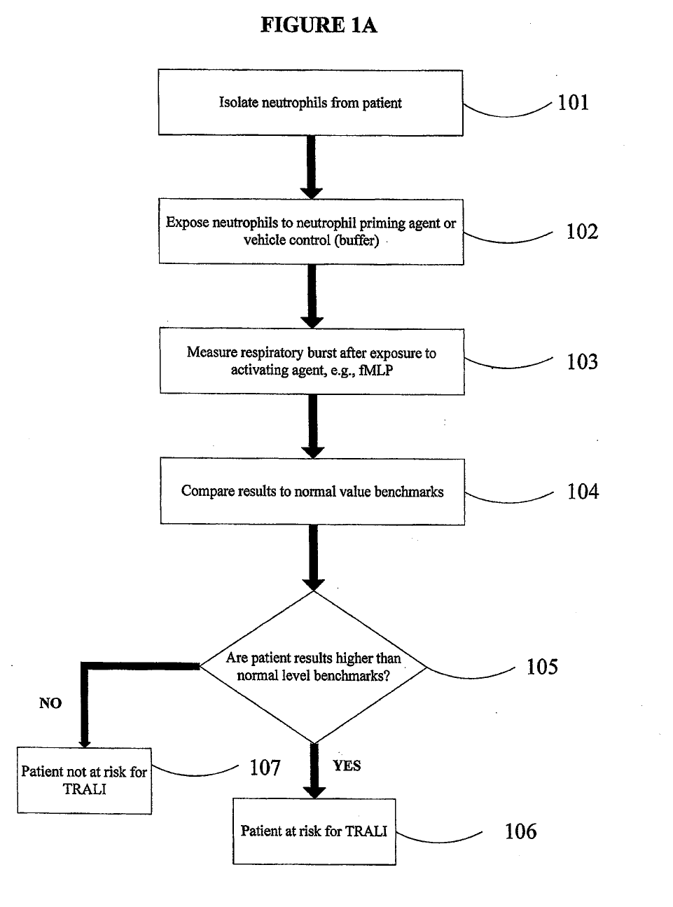

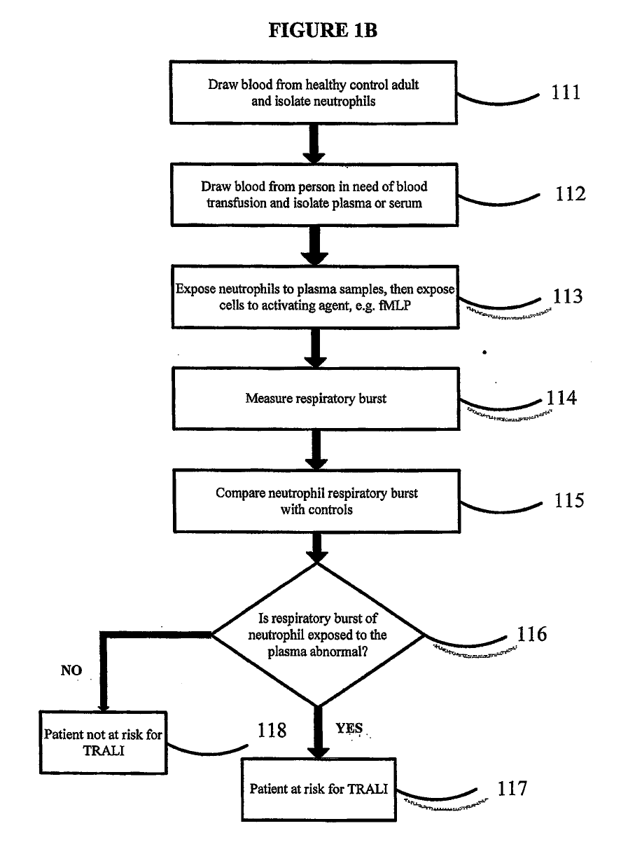

A first embodiment as depicted in FIG. 1A is to a method for testing priming of neutrophils. First, blood is drawn from a healthy test subject or patient in need of a blood transfusion and the neutrophils are isolated (101). The neutrophil isolation may occur by any standard technique, including but not limited to, dextran sedimentation, Ficoll Hypaque gradient centrifugation, and hypotonic lysis of contaminating red cells; other similar techniques separating neutrophils by their buoyant density; or even FACS sorting when a suitable fluorescence tag is associated with a neutrophil or class of neutrophils.

Expose the neutrophils to a neutrophil priming agent (102) or appropriate blood sample. The neutrophil priming agent used is not limiting, and therefore may be natural or synthetic, and includes fragments, analogues, and domains of any neutrophil priming agent.

The specific condition of the neutrophil exposure to the neutrophil priming agent will depend upon the exact priming agent used. Further, the time that the neutrophils are exposed to the neutrophil priming agent will depend on the priming agent used. For example, IL-18 and other cytokines or chemokines primes neutrophils but does so over a 15-60 minute time frame. LPS primes over 30 minutes. On the other hand, sCD40L, lyso-PCs, LTB₄, or antibodies to HNA-3a all prime the neutrophils within 5 minutes.

Subsequent exposure of the neutrophils to an neutrophil activating agent, such as, for example, fMLP, results in a respiratory burst, and the compounds or ROS released by the neutrophils may then be measured (103). Thus, it is contemplated that a skilled practitioner in the arts may measure superoxide anions, hydroxyl radicals, hydrogen peroxide and/or singlet oxygen. The contemplated methods of measuring the respiratory burst compounds are discussed in detail below.

The respiratory burst compounds or ROS measured are then compared to a benchmark “normal level” respiratory burst compounds or ROS levels in response to neutrophil activating agent alone. In other words, the “benchmark” can be the respiratory burst compounds or ROS levels releases from neutrophils treated with a neutrophil priming agent and/or activating agent, where the neutrophils were obtained from normal, control patients who does not have TRALI. The benchmark may be different for sub-populations. For instance, the “benchmark” may vary by age, race, gender, genetic dipositions (such as the presence or absence of SNPs), and the like. The comparison of any respiratory burst compounds or ROS levels obtained may be performed by visual confirmation of, for instance, a fluorophore. Alternatively, the comparison may performed by a computer-based system comprising a data storage means having stored information regarding respiratory burst compounds or ROS levels of TRALI and non-TRALI patients, with the necessary hardware means and software means for comparing and saving any comparisons between patient data and/or patient and benchmark data. The software means of the computer-based system includes one or more software programs or algorithms that are implemented on the computer-based system to identify respiratory burst compounds or ROS levels based on respiratory burst compounds or ROS levels information stored within the data storage means. Thus, the practitioner may provide the computer-based system with information regarding the respiratory burst compounds or ROS levels on a computer readable medium, including but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. Further, data processor programs and formats can be used to store the respiratory burst compounds or ROS levels information of the present invention on computer readable medium, such as word processing text file, commercially-available software such as WordPerfect and Microsoft Word, ASCII files, or stored in a database application, such as OB2, Sybase, Oracle, or the like. A skilled artisan can readily adapt any number of data processor structuring formats (e.g., text file or database) in order to obtain computer readable medium having recorded thereon information regarding respiratory burst compounds or ROS levels. The respiratory burst compounds or ROS levels information of the present invention may be stored/analyzed in a computer-based system for later determining or analyzing potential benchmarks of TRALI, respiratory burst compounds or ROS levels of interest, frequencies in a population, correlating respiratory burst compounds or ROS levels of a patient with a benchmark, or other patients, or for various other bioinformatic, pharmacogenomic, or drug development applications. By use of such computer systems, or by personal visual confirmation, the practitioner will then have to determine if the results are higher than the normal level benchmark (105). If the level of respiratory burst in the test is higher than a normal level, i.e., abnormally high, then the TRALI reaction is associated with a neutrophil priming activity or the patient is considered at-risk for TRALI (106). If the value of the respiratory burst is close to or below a normal value, then the TRALI reaction is not associated with neutrophil priming or the patient is considered not at-risk for TRALI (107).

The comparison of the patient results with the normal level benchmark may occur by any means, including algorithm analysis, use of statistics to find statistical differences in values.

For example, in one embodiment the baseline level of the respiratory burst stimulated with fMLP alone is defined as less than about 3 nmol/min/10⁶ cells/ml of superoxide or equivalent as determined by other techniques. In one embodiment, significant priming is defined as >about 1.5 times the level expressed by patient cells in response to fMLP or another neutrophil activating agent, or by test neutrophils exposed to specific neutrophil activating agent or sample then to fMLP. In another embodiment, significant priming is defined as >about 2.0 times the level expressed by patient cells in response to fMLP or another neutrophil activating agent, or by test neutrophils exposed to specific neutrophil activating agent or sample then to fMLP. This may be expressed as absolute rate or ratio over baseline.

Another embodiment of the invention depicted in FIG. 1B is to a method for testing of priming agents in the plasma of a person in need of a blood transfusion. First, blood is drawn from a healthy test subject or patient in need of a blood transfusion and the plasma is isolated (111). The plasma may be isolated from the cells of the blood by any means, including centrifugation or sedimentation.

Neutrophils are then isolated from the blood of a healthy control adult (112). The neutrophil isolation may occur by any standard technique, including but not limited to, dextran sedimentation, Ficoll Hypaque gradient centrifugation, and hypotonic lysis of contaminating red cells; other similar techniques separating neutrophils by their buoyant density; or even FACS sorting when a suitable fluorescence tag is associated with a neutrophil or class of neutrophils.

The neutrophils should be parsed into separate containers so experiment controls may be set. For instance, one set of neutrophils from the healthy control adult are exposed to the plasma of the person in need of a blood transfusion (113). Another set of neutrophils serve as a control, and are only exposed to buffer.

At this same time, one or more additional sets of neutrophils may be exposed to various concentrations of one or more neutrophil priming agents. The neutrophil priming agents used are not limiting, and therefore may be natural or synthetic, and includes fragments, analogues, and domains of any neutrophil priming agent. The specific condition of the neutrophil exposure to the neutrophil priming agent will depend upon the exact priming agent used. Further, the time that the neutrophils are exposed to the neutrophil priming agent will depend on the priming agent used. For example, IL-18 and other cytokines or chemokines primes neutrophils but does so over a 15-60 minute time frame. LPS primes in 30 minutes. On the other hand, sCD40L, lyso-PCs, LTB₄, or antibodies to HNA-3a all prime the neutrophils within 5 minutes.

The respiratory burst of the neutrophils may then be measured by detection of compounds or ROS released by the neutrophils (114). Thus, it is contemplated that a skilled practitioner in the arts may measure superoxide anions, hydroxyl radicals, hydrogen peroxide and/or singlet oxygen. The contemplated methods of measuring the respiratory burst compounds are discussed elsewhere within the specification.

The respiratory burst compounds or ROS measured are then compared to a control respiratory burst compounds or ROS release (115). For example, the respiratory burst compounds may be compared to a control of neutrophils in buffer, neutrophils exposed to neutrophil priming agents, and the like. The practitioner will then have to determine if the respiratory burst of the neutrophils exposed to the neutrophil priming agent is abnormal (116). If the level of respiratory burst in the neutrophils exposed to the plasma of a person in need of a blood transfusion is higher than a normal level, i.e., controls, then the practitioner may determine the plasma contains neutrophil priming agents. Thus, the patient is considered at-risk for TRALI (117). If the level of respiratory burst in the neutrophils exposed to the plasma of a person in need of a blood transfusion is close to or below a normal value, then the patient is considered not at-risk for TRALI (118).

The comparison of the patient results with the normal level benchmark may occur by any means, including algorithm analysis, use of statistics to find statistical differences in values.

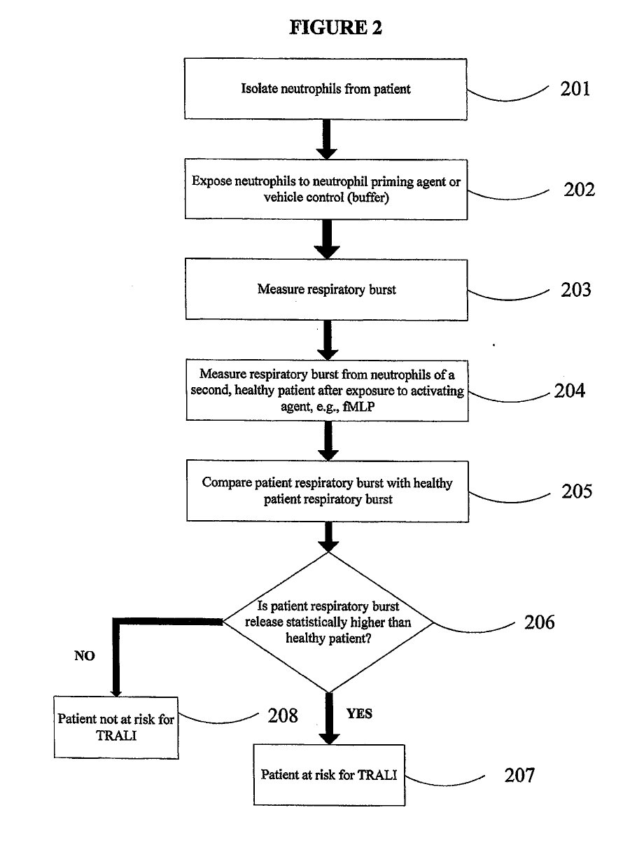

Another embodiment of the invention is depicted in FIG. 2 for a method for testing the priming state of the neutrophils of a patient in need of a blood transfusion. First, blood is drawn from a patient in need of a blood transfusion and the neutrophils are isolated (201). The neutrophil isolation may occur by any standard technique such as those described above, including but not limited to, dextran sedimentation, Ficoll Hypaque gradient centrifugation, and hypotonic lysis of contaminating red cells.

Expose the neutrophils to a neutrophil priming agent (202). The neutrophil priming agent used may include, but is not limiting, and therefore may be natural or synthetic, and includes fragments, analogues, and domains of any neutrophil priming agent. The specific conditions and timing of the neutrophil exposure to the neutrophil priming agent will depend upon the exact priming agent used.

The exposure of the neutrophils to the neutrophil priming agent results in a respiratory burst, and the compounds released by the neutrophils may then be measured (203). Thus it is contemplated that a skilled practitioner in the arts may measure superoxide anions, hydroxyl radicals, hydrogen peroxide and/or singlet oxygen. The contemplated methods of measuring the respiratory burst compounds are discussed in detail below.

The neutrophils of a healthy patient are then exposed to the same neutrophil priming agent in the exact same conditions as in step (203), whereby the respiratory burst release is measured in the healthy patient (204). The respiratory burst release of the healthy patient is then compared to the respiratory burst release of the patient in need of a blood transfusion (205). The comparison can occur by any means, including use of computer algorithms, and the like.

The practitioner will then determine if the respiratory burst compounds released from the neutrophils of the patient in need of a blood transfusion is statistically higher than the healthy patient respiratory burst release (106). If the level of respiratory burst compounds in the patient in need of a blood transfusion is statistically higher than the healthy patient respiratory burst, i.e., abnormally high, then the patient is considered at-risk for TRALI (207). If not, then the patient in need of a blood transfusion is not at-risk for TRALI (208).

### Neutrophil Priming Agents

The neutrophil priming agent may be any agent that alone does not induce a respiratory burst but enhances the activity in response to an activating agent such as fMLP compared to the stimulation by the activating compound alone. Priming agents include, but are not limited to, bioactive lipids such as leukotriene B₄ (LTB₄) and lysophosphatidylcholines (lyso-PCs), cytokines or chemokines such as interleukin-18 (IL-18) or granulocyte colony stimulating factor (G-CSF), donor antibodies specifically directed against recipient leukocytes, lipid soluble CD40 ligand (sCD40L), tumor necrosis factor (TNF), chemotactic peptide fMLP (at lower concentrations than used as an activating agent), 4-phorbol-12-β-myristate-13-acetate, antibodies to the HNA-3a locus, and vascular endothelial growth factor (VEGF). The neutrophil priming agent used is not limiting, and therefore may be natural or synthetic, and includes fragments, analogues, and domains of any neutrophil priming agent. The specific conditions and timing of the neutrophil exposure to the neutrophil priming agent will depend upon the exact priming agent used. Many of these compounds are found in stored blood components or blood from patients with certain disease states.

### Measurement of Respiratory Burst

Neutrophil priming results in enhancement of the “respiratory burst” stimulated by a standard activating agent such as fMLP causing release of ROS consisting of superoxide anion, hydroxyl radical, hydrogen peroxide and singlet oxygen. Any of these compounds may be measured to indicate the level of neutrophil activation.

Singlet oxygen may be measured by any means convenient to the practitioner, including but not limited to, use of fluorescent, chemiluminescent singlet oxygen detection reagents and photo oxidation of 1,3-diphenylisobenzofuran. In one embodiment, singlet oxygen is measured by use of Singlet Oxygen Sensor Green reagent (Invitrogen; California). Before reaction with singlet oxygen, the reagent initially exhibits weak blue fluorescence with excitation peaks at 372 and 393 nm and emission peaks at 395 and 416 nm. In the presence of singlet oxygen, however, it emits a green fluorescence similar to that of fluorescein (excitation/emission maxima ˜504/525 nm).

Hydroxyl radical may be measured by any means convenient to the practitioner, including but not limited to, use of spin traps, chromophores or other enzyme reactions. In this embodiment, fluorogenic probes are used to detect hydroxyl radicals, which may be used to measure hydroxyl radicals by fluorescence or by electron spin resonance spectroscopy. Two examples of fluorogenic probes for hydroxyl radicals are TEMPO-9-AC and proxyl fluorescamine, which contains a nitroxide moiety that quenches its fluorescence. Alternatively, hydroxyl radical may be detected by release of ethylene gas in a chemical assay as measured by gas chromatography.

Superoxide anion may be measured by any means convenient to the practitioner, including but not limited to, chemiluminescent and chromogenic reagents. Examples of appropriate chemiluminescence reagents includes, but are not limited to, coelenterazine (Invitrogen; California), MCLA (Invitrogen; California), and lucigenin, digenes. Chromophores which are reduced include nitro blue tetrazolium or cytochrome c. Further, electron spin resonance may be used to measure superoxide anion, including use of pH-jump, which is a simple operation for trapping O₂⁻ without the use of any rapid-mixing apparatus.

Hydrogen peroxide may be measured by any means convenient to the practitioner, including but not limited to, fluorescent or chemiluminescent reagents. In one embodiment, hydrogen peroxide is measured by use of Amplex Red reagent (10-acetyl-3,7-dihydroxyphenoxazine) with horseradish peroxidase, producing a red-fluorescent product for colorimetric determination. Hydrogen peroxide can be measured by oxidation of dihydrorhodamine or other fluorescent probe in a flow cytometer.

The use of spin traps/electron paramagnetic resonance spectroscopy (EPR) is one approach for detecting superoxide anion and other reactive oxygen species. Specific spin traps include, but are not limited to, 5,5 dimethyl-1-pyrroline N-oxide 1; 5-(diethoxyphosphoryl)-5-methyl pyrroline N-oxide 2; 5-tert-butoxycarbonyl-5-methyl-1-pyrroline N-oxide; 2-diemoxyphosphoryl-2-phenemyl-3,4-dihydro-2H-pyrrole N-oxide; or 5-diemoxyphosphoryl-5-methyl-1-pyrroline N-oxide. Here, the spin traps are added to the assay mixture with isolated neutrophils which are primed with plasma or blood samples previously noted or with other biologically active compounds and stimulated with fMLP (1 μM). The specific spin adducts may be determined with EPR spectroscopy and the ratio of adducts produced with priming and activation calculated with the results with the fMLP stimulus alone. This defines the priming activity for the specific plasma sample or biologically active agent. Other traps may be used to determine the effect on hydrogen peroxide, hydroxyl radical and other reactive oxygen species.

The priming activity of plasma specimens from various sources including patient samples and blood components has been described herein. The effects on priming by various dilutions may correlate with the concentration of biologically active compounds in the specimens. Many of these compounds are well characterized and their priming activities determined. For example, lysophosphatidylcholines with 1-O-Palmitoyl, 1-O-Oleoyl, 1-O-Stearoyl, 1-O-Lauroyl, 1-O-Myristoyl and other 16-18 carbon fatty acid groups on the SN-1 position exhibit priming activity when added to fatty acid free albumin and tested for priming of the neutrophil oxidase in the systems described. Each has its own concentration dependent effects. IL-8 and TNF-alpha are among other cytokines, chemokines and growth factors found in biologically active plasma samples which exhibit concentration dependent priming of the fMLP stimulated respiratory burst. Similar effects are seen with complement fragments (e.g., C5a), other biologically active lipids (e.g., leukotrienes), and other compounds (e.g., sCD40L). Antibodies to neutrophil antigens (e.g., HNA-3a) also can prime the oxidase enzyme system. All these agents may be detected in plasma from blood components implicated in TRALI or plasma from patients sustaining a TRALI reaction. All may be added directly to the assay and priming activity detected as described in other sections.

In one preferred embodiment, respiratory burst is measured by use of cytochrome c. There are multiple methods for measuring cytochrome c, including use of immunochemical methods, and methods utilizing electrophoresis. Examples of the methods utilizing electrophoresis include a method wherein polyacrylamide gel electrophoresis is performed to detect cytochrome c as a band, a method wherein capillary electrophoresis is performed to detect cytochrome c as a peak and so forth. Further, one embodiment is to a method utilizing chromatography such as high performance liquid chromatography to detect cytochrome c as a peak. To increase sensitivity, fluorescence labeling may also be used. Further, the levels of cytochrome c in the embodiments are quantifiable.

In one preferred embodiment, the method for measuring cytochrome c is an immunochemical method. Such methods tend to have high sensitivity and are simple to conduct. The term “immunochemical method” used herein refers to a method of determining cytochrome c by using an antibody directed to cytochrome c. Immunochemical methods to measure cytochrome c include, but are not limited to, competitive methods in which cytochrome c is labeled, sandwich methods in which an antibody is labeled, a latex bead method in which agglutination of antibody-coated beads is observed, and the like. The antibody to cytochrome c may be a monoclonal or polyclonal antibody. It is also possible to label with a radioactive isotope, with a compound showing electro-chemiluminescence, fluorescence labeling, label with an enzyme, label with biotin, and the like.

The present invention also relates to a reagent for testing for TRALI, which comprises a reagent for determination of cytochrome c levels. The reagent for determination of cytochrome c is preferably one for determination by an immunochemical method. An example of the reagent is an antibody directed to cytochrome c.

In one embodiment, the immunochemical method is a sandwich method. The sandwich method is an immunochemical method such as ELISA utilizing an antigen sandwiched by an immobilized antibody and a labeled antibody.

Further, one embodiment is to compositions comprising a test reagent used for measuring cytochrome c in body fluid by the sandwich method, which comprises an antibody directed to cytochrome c as an ingredient. This measurement reagent may have the same constitution as that of a reagent (kit) used in a usual sandwich method except that the anti-cytochrome c antibody is used as an antibody. For example, the reagent for measuring cytochrome c by the sandwich method may contain 1) an anti-cytochrome c antibody-coated solid phase such as an anti-cytochrome c antibody-coated cup or anti-cytochrome c antibody-coated beads, 2) a labeled anti-cytochrome c antibody, 3) a cytochrome c standard solution of a known concentration, 4) a diluent and 5) a washing solution. Further, if labeling with an enzyme is used, 6) a chromogenic substrate and 7) a solution for terminating a reaction may be included.

### Kits and Assays

Embodiments of the present invention also include to diagnostic kits and assays used to determine levels of respiratory burst. Such a kit would include means for detecting presence of a respiratory burst compound, positive and/or negative control reagents, and instructions for determining the levels of respiratory burst with the kit.

The kit of the present invention also provides components for carrying out methods of the invention. Accordingly, in one embodiment, the kit further contains means for measuring the level of the respiratory burst. The kit may further contain one or more neutrophil priming agents. Further, the kit may contain appropriate containers, such as 96-well plates.

In one embodiment, a kit is provided that uses luminophoric reagents to quantify production of reactive oxygen species including; superoxide anion, hydrogen peroxide and hydroxyl radical. In this embodiment, the kit may one or more of the following: a luminophoric reagent such as, for example, luminal, buffer, positive and negative control reagents, and a 96-well plate.

Immunoassay techniques useful for the present invention and well known to those of skill in the art include, but are not limited to, radioimmunoassays, techniques employing magnetic separation and electrochemiluminescent measurement applied to detection antibodies labeled, for example, with rubidium, immunoprecipitation, Western blot analysis (immunoblotting), and fluorescence-activated cell sorting (FACS).

Design of the immunoassays is subject to a great deal of variation, and many formats are known in the art. Protocols may, for example, use solid supports, or immunoprecipitation. Many assays involve the use of labeled antibody or polypeptide; the labels may be, for example, enzymatic, fluorescent, chemiluminescent, radioactive, or dye molecules. Assays which amplify the signals from the immune complex are also known; examples of which are assays which utilize biotin and avidin, and enzyme-labeled and mediated immunoassays, such as ELISA assays.

The immunoassay may be, without limitation, in a heterogenous or in a homogeneous format, and of a standard or competitive type. Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride (known as Immulon), diazotized paper, nylon membranes, activated beads, and Protein A beads. For example, Dynatech Immulon1 or Imnulon2 microtiter plates or 0.25 inch polysterene beads (Precision Plastic Ball) can be used in the heterogeneous format. In a homogeneous format, the test sample is incubated in solution. For example, it may be under conditions that will precipitate any antigen-antibody complexes which are formed. Both standard and competitive formats for these assays are known in the art.

Further, respiratory burst may be measured quantitatively by fluorometric analysis by flow cytometry. One such embodiment is described below.

When used here, “about” generally means +/−10% of the number; such that, for example, “about 10 to about 20” refers to a range of 9-11 to 18-22.

### EXAMPLE 1

Whole blood is drawn from healthy donors after obtaining informed consent. Neutrophils are isolated by standard techniques including dextran sedimentation, Ficoll-Hypaque gradient centrifugation, and hypotonic lysis of contaminating red blood cells. Neutrophils are then be resuspended in warm (37° C.) Krebs-Ringers-Phosphate buffer with 2% dextrose (KRPD). The maximal rate of superoxide anion production in response to fMLP (10⁻¹² to 10⁻⁶ M) or to PMA (200 ng/ml) is then measured by the SOD-inhibitable reduction of cytochrome c at 550 run at 37° C. The priming activity of the agent or test plasma is completed by first incubating the neutrophils in the reaction mixture with the priming agent or test plasma for 3 min at 37° C. followed by activation of the oxidase with the addition of fMLP or PMA. Therefore, priming activity in these experiments is measured as the augmentation of the rate of superoxide anion production in response to fMLP or PMA.

### EXAMPLE 2

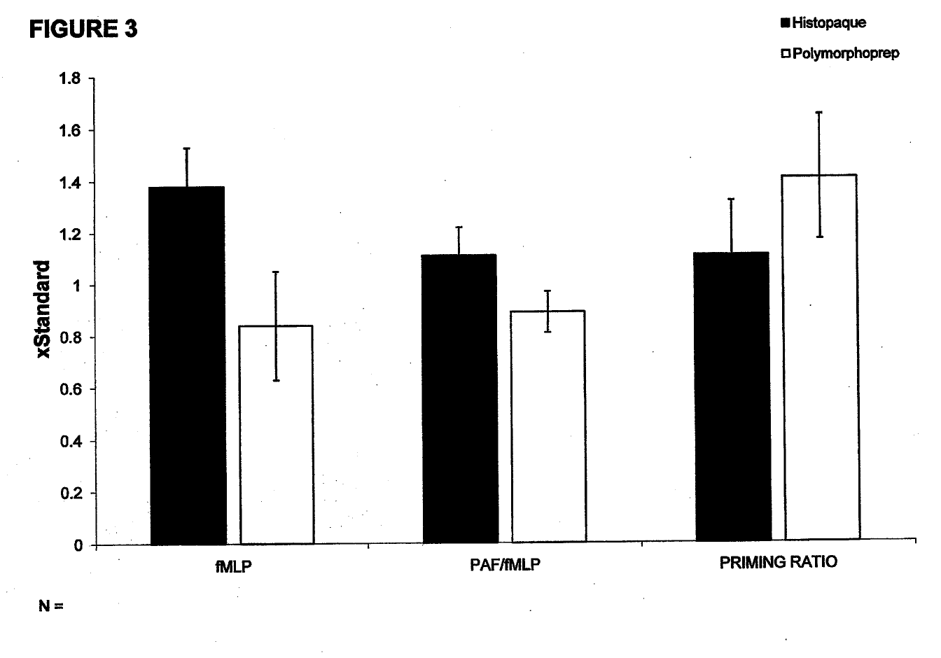

For the assays, cells used as targets include neutrophils isolated from peripheral blood, cultured myeloid cell lines matured into neutrophil-like cells, and cytoplasts. Neutrophils may be isolated from peripheral blood more rapidly using one step gradients than dextran sedimentation and Ficoll-Hypaque density gradient centrifugation. Comparative studies using Histapaque and Polymorphoprep single step gradients and our standard procedure demonstrated a decrease in time of preparing target neutrophils to 30 minutes. FIG. 3 summarizes results of a series of studies measuring superoxide anion production by neutrophils in response to fMLP (1 μM) and PAF/fMLP (1 μM/1 μM) as cytochrome c reduction. The results for Histapaque (shaded bars) and Polymorphoprep (hatched bars) isolated neutrophils are expressed as a ratio for results with neutrophils isolated by our standard procedure. The Histapaque neutrophils generated increased superoxide anion with both fMLP and PAF/fMLP; the Polymorphoprep cells were quieter, but exhibited a better ratio of PAF/fMLP to fMLP alone (priming ratio, third set of bars). These studies demonstrated that the Polymorphoprep provided a rapid, one-step procedure to isolate resting neutrophils to be used as targets for the priming assays.

Cytoplasts are neutrophils which have been sedimented on a 20% Ficoll gradient at 77,000×g. Cytoplasts, are devoid of nucleus, granules and other subcellular organelles. Cytoplasts retain the ability for random and directed migration, phagocytosis of opsonized particles as well as activation of a respiratory burst and generation of reactive oxygen species. They can be frozen at −70° C. and thawed; the thawed cytoplasts can be used as targets for priming activity. The advantage is that cytoplasts would be readily available for assaying priming activity of biologic specimens (FIG. 1B, 113) in place of freshly isolated neutrophils from a healthy donor. Studies optimizing use of cytoplasts for generation of superoxide anion measured as cytochrome c reduction demonstrate the following characteristics:
a) Optimum concentration 7.5×10⁶ cytoplasts/ml in the reaction volume
b) Concentration dependent activation (1 μM fMLP) and priming by PAF (1 μM) of the fMLP stimulated respiratory burst are identical to intact neutrophils
c) Priming of cytoplasts by PAF enhances the fMLP stimulated respiratory burst by 1.6 to 2.0 fold
d) Plasma from Day 7 stored platelets primes the fMLP stimulated respiratory burst compared to plasma from Day 0 stored platelets

These data confirm the utility of cytoplasts as a target for priming in place of intact neutrophils.

Finally, cultured myeloid cells, such as HL-60 cells, may be matured in culture with the addition of DMSO or retinoic acid. The resultant maturation provides cells with characteristics similar to those of peripheral neutrophils. Specifically, mature HL-60 cells exhibit a complete NADPH oxidase and respiratory burst which may be primed by PAF, lipids, cytokines, chemokines and other biologically active compounds found in stored blood components and implicated in TRALI.

### EXAMPLE 3

Assay of the respiratory burst and cytochrome c reduction has been described previously but has been simplified to determine priming in a simple procedure that does not require a washing step and is complete in a shorter time. In this procedure, the biologic specimen to be analyzed for priming activity is added to target cells (neutrophils, cytoplasts or cultured myeloid cells), incubated for 5 minutes and fMLP is introduced to activate the target cells. Superoxide anion is determined by SOD inhibitable cytochrome c reduction. The assay system has been optimized to contain the following additions kept under specific conditions.
a) Neutrophils, 3.75×10⁵ cells per well at cell concentration 2.5×10⁶/ml
b) Cytochrome c (75 μM)
c) SOD to blank (15 μg/ml)
d) Stimulus, fMLP (1 μM)
e) Biological priming sample: PAF (1 μM); 15 μl plasma or serum; cytokine, chemokine, lipid or biologically active compound in suitable buffer; or buffer control.
f) Total volume 150 μl

Reduction of cytochrome c determined in an ELISA plate reader at 550 nm using SOD blank to determine initial rate of superoxide anion in response to fMLP.

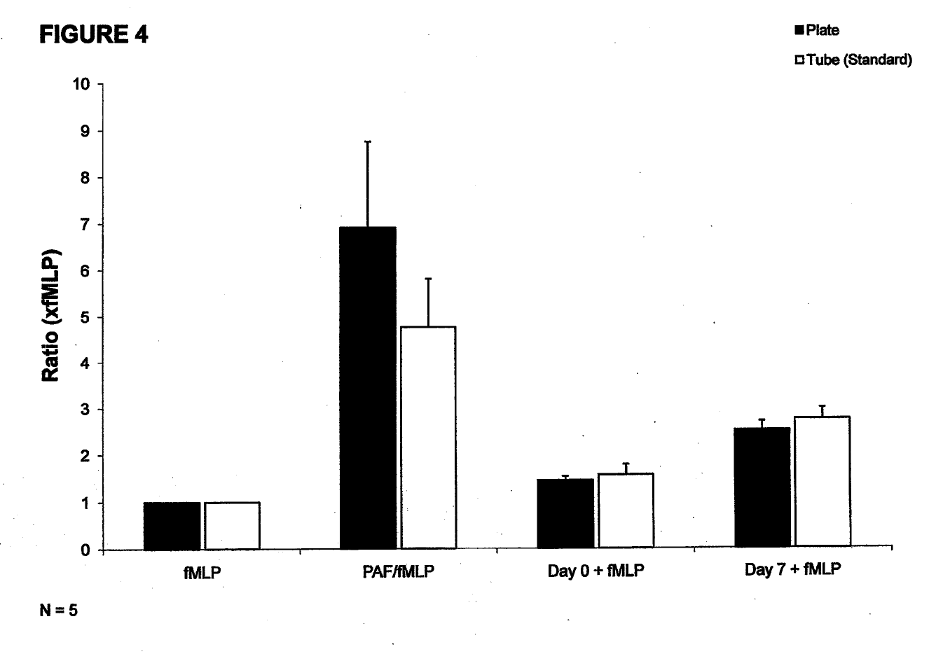

The simplified system is validated to standard assay with a centrifugation step to remove priming sample from neutrophils. Results for 5 different experiments evaluating priming of neutrophils with PAF (1 μM), five different plasma samples (10% by volume) from platelet concentrates at Days 0 and 7 of storage are shown in FIG. 4. The enhancement of the fMLP response is expressed as a ratio of superoxide anion produced with fMLP alone. The shaded bars represent the standard procedure and hatched bars the procedure without washing, termed “plate” assay. As can be seen the plate assay shows that plasma from Day 0 stored platelets has very little priming activity (<1.5 times the fMLP response alone). In contrast, stored platelet concentrates contain, in the plasma phase, biologically active compounds which prime the fMLP response >two fold over fMLP stimulation alone. In addition, a product which clinically was associated with a TRALI reaction was associated with significant priming. This plate assay which can provide comparable priming results to the standard assay may be completed within 45 minutes to one hour, an important technical advance for clinical laboratories.

### EXAMPLE 4

### Priming Assay Using Fluorescent Probe for Hydrogen Peroxide

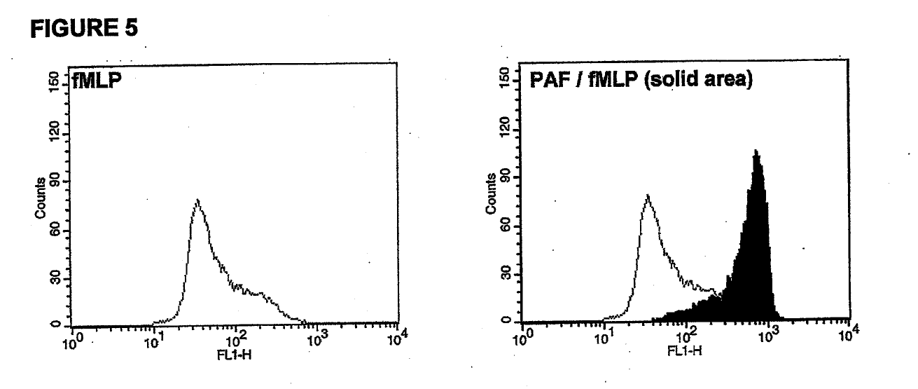

Hydrogen peroxide (H₂O₂) is another reactive oxygen species produced by neutrophils when the NADPH oxidase and associated respiratory burst are activated. H₂O₂ can be detected intracellularly by incubating cells with dihydrorhodamine (DHR). This non-fluorescent compound will be oxidized to rhodamine, a potent fluorophore, when H₂O₂ is generated by activated neutrophils. This assay can be used to detect enhancement of the fMLP stimulated respiratory burst. FIG. 5 demonstrates the characteristics of the assay. Isolated peripheral blood neutrophils are incubated with 50 μM DHR. After exposure to fMLP (1 μM) or PAF (1 μM) for 3 minutes and fMLP (1 μM), the cells are placed on ice and samples analyzed with the evaluation of 10,000 events on a BD Canto flow cytometer. FIG. 5 demonstrates the flow scan as a plot of cell counts vs. fluorescence. fMLP by itself results in a modest respiratory burst with a mean channel fluorescence of 45. When PAF is used to prime the fMLP response, the result is a marked increase in H₂O₂ production expressed as MCF (˜250 MCF units).

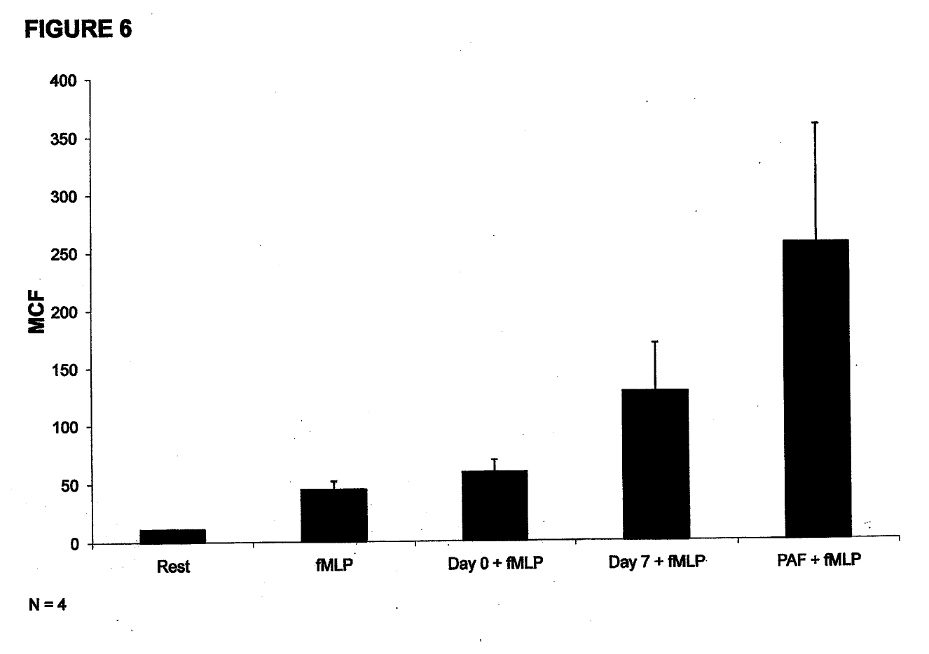

This assay has been further optimized for the following reagents: DHR, 50 μM with the best loading time of 1 minute. Optional concentration of fMLP is 1 μM and PAF 1 μM. The assay should be analyzed in a flow cytometer as soon as possible but may be delayed up to 4 hours after completion without jeopardizing the results. Finally, plasma itself quenches the fluorescence. As a result the priming assay on this platform requires exposure of the cells to buffer or test sample for priming (PAF, plasma, etc), a low speed centrifugation to pellet cells, resuspension of cells in DHR containing buffer, then stimulation with fMLP. Determination of MCF is by analysis of 20,000 events. FIG. 6 summarizes results, expressed as mean ±SEM, with this assay in four separate experiments. Resting cells with buffer present very low levels of H₂O₂ with an increase when resting cells are stimulated with fMLP. Plasma from Day 0 and 7 stored platelet concentrates has little effect in the absence of fMLP. Plasma from Day 0 stored platelet concentrates enhances the fMLP stimulated respiratory burst only slightly. The priming of the fMLP stimulated burst is markedly enhanced by plasma for Day 7 stored concentrates and PAF. Thus, the fluorescent assay is suitable to demonstrate priming.

### EXAMPLE 5

### Detection of Priming by Chemiluminescence

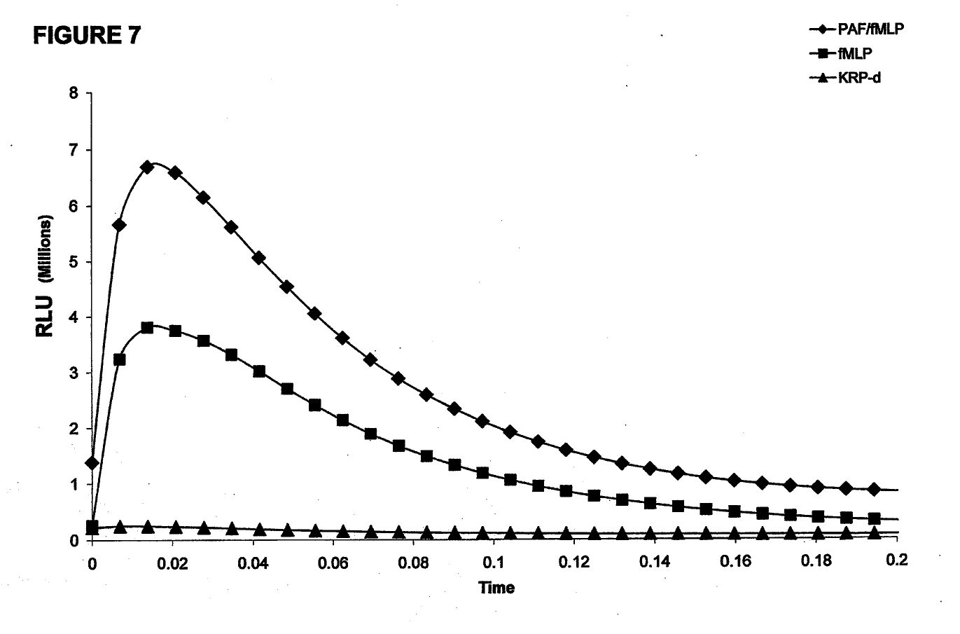

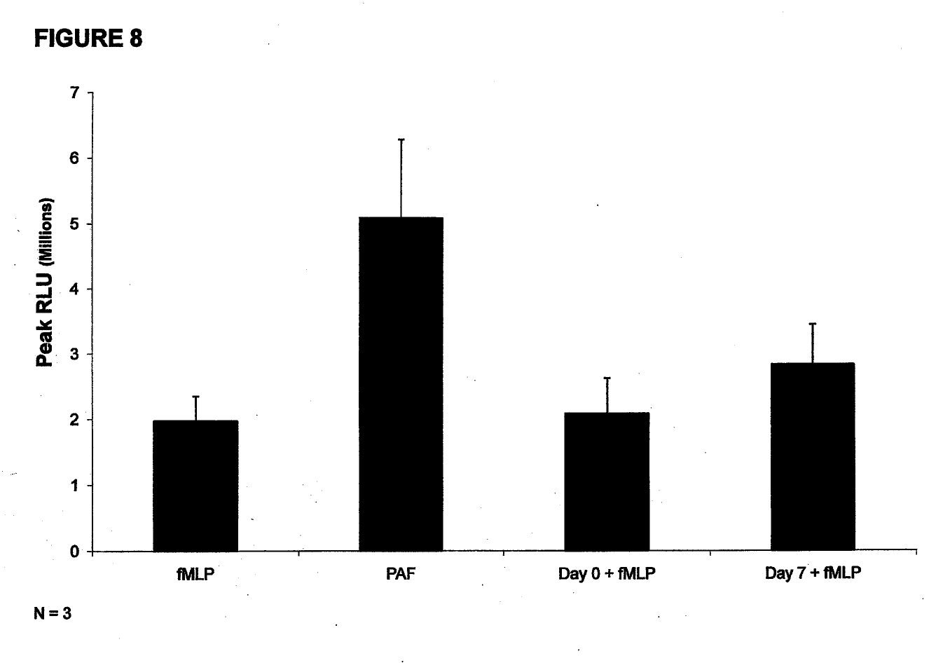

A third assay makes use of energy from chemiluminescence to determine oxygen radical generation from activated neutrophils. In this reaction, neutrophils are incubated with the priming sample or buffer, then exposed to fMLP after addition of the chemiluminescent probe, Diogenes, and the energy released determined in a chemiluminescence reader. The optimal cell concentration is between 2.5×10⁶ cells/ml and 5×10⁶ cells/ml. The chemiluminescence is >90% SOD inhibitable and so detects superoxide anion. The optimal addition of diogene is 20 μl to provide specific determination of oxygen radicals released by the neutrophils respiratory burst. FIG. 7 demonstrates chemiluminescence in response to fMLP alone and PAF priming of the fMLP response. Buffer alone is included for control. The PAF primed response is represented by both a higher level of relative light units (RLUs) as well as a more prolonged time course at maximal production. As with fluorescence platform, plasma nonspecifically quenches the chemiluminescence. This can be circumvented with an extra wash step to remove plasma after incubating cells with the priming stimulus. The resultant assay is sensitive to the effect in stored blood products. FIG. 8 demonstrates the effect of priming of the fMLP response by platelet concentrates during storage in three separate experiments. Plasma from Day 0 stored platelet concentrates do not enhance the fMLP burst. However, Day 7 primes the fMLP stimulated chemiluminescence. The priming effect for PAF is presented for comparison. The results are expressed as mean ±SEM. This platform, using chemiluminescence, can be used to determine priming by biologic samples.

### EXAMPLE 6

The neutrophil priming agent sCD40L is synthesized or isolated in advance, employing standard techniques. In short, the insect cell line Sf-21 is grown in suspension culture in Hy-Q SFX (Hyclone) serum-free insect cell culture medium supplemented with Pluronic F-68 surfactant. The cells are infected for 2 to 3 days with a recombinant baculovirus encoding full length human CD 154 (hCD154), harvested, washed in Gey's solution, and homogenized using a Polytron homogenizer. The cell homogenate is then centrifuged on a sucrose gradient at 90,000×g to isolate the cell membrane fraction, which will then be harvested and washed extensively with phosphate buffered saline solution to yield a suspension of insect cell membranes expressing hCD1 54 protein. Western blotting the preparation and detecting the appropriate ≈31-33 kDa protein is then performed to confirm the presence of hCD1 54 on the insect cell membranes. Bioactivity of the hCD1 54 can be confirmed by dose-responsive activation of normal human lung fibroblasts expressing CD40 to produce IL-8 in culture.

Neutrophils are isolated by standard techniques including dextran sedimentation, Ficoll-Hypaque gradient centrifugation, and hypotonic lysis of contaminating red blood cells.

The neutrophils are warmed to 37° C., [10%]_{FINAL} plasma is added, and the neutrophils are incubated for 5 minutes at 37° C. The neutrophils are then washed, resuspended in warm Krebs-Ringers-Phosphate with 2% dextrose, pH 7.35 (KRPd), and pipetted into a 96 well place. 80 μM cytochrome c is then added to the mixture.

The monomeric recombinant sCD40L will then be added to the wells of the 96-well place in a concentration of 10 to 1,000 ng/ml. After approximately five minutes, the neutrophil respiratory burst oxides can be determined by cytochrome c detection via ELISA reader at 550 nm. The results of measuring the neutrophil respiratory burst can then be compared to that of a healthy subject, to find if the patient is at-risk for TRALI.

### EXAMPLE 7

Neutrophils are isolated from heparinized peripheral blood, washed, are resuspended in warm Krebs-Ringers-Phosphate with 2% dextrose, pH 7.35 (KRPd) buffer, are added to the reaction mixture containing 80 μM cytochrome c or in selected wells containing 15 μg/ml superoxide dismutase (SOD), and the maximal rate of the reduction of cytochrome c is measured at 550 nm. The SOD controls are employed such that the data reflects the maximal rate of SOD-inhibitable reduction of cytochrome c(nmol/min). Positive controls, neutrophils primed with 2 μM platelet activating factor (P AF), and negative controls, buffer and fresh plasma primed neutrophils, are included on every 96-well reaction plate. Leukotriene B₄ (LTB₄) is then added to induce respiratory burst from the neutrophils. The respiratory burst of the neutrophils are measured and compared to a threshold “normal level” respiratory burst level, to find if the patient has neutrophils with advanced respiratory burst levels. Alternatively, serum or plasma from blood components or patients sustaining TRALI can be used in priming phase and compared to normal serum or plasma to determine the fMLP stimulated respiratory burst.

### EXAMPLE 8

Neutrophils are isolated from a patient in need of a blood transfusion. They are then washed and resuspended in 50 mM phosphate-buffer (pH 7.4). Lysophosphatidylcholines (lyso-PCs) is then be added to the neutrophils. After 5 minutes of equilibration, the cell suspensions is centrifuged for 1 min at 6,000 rpm at 4° C. and the supernatant removed from the cells. The H₂O₂ concentrations is then measured by the Amplex Red Hydrogen Peroxide Assay Kit (Molecular Probes Inc., USA), which contains a highly sensitive and specific fluorogenic probe (U-acetyl-3,7-dihydroxyphenoxazine) for H₂0₂ and horseradish peroxidase (HRP). Briefly, 100 μl supernatant is mixed with 100 μl of the probe at 100 μM and 1 U/ml HRP. The fluorometric assay is conducted in a 96-well microplate and measured by Luminescence Spectrometer LS50B (Perkin Elmer). The excitation wavelength is set at 540 ran; the emission wavelength is measured at 590 nM. The concentrations will then be calculated based on the H₂0₂ standard curves generated simultaneously. If the H₂O₂ released from the neutrophils exceeds a predetermined threshold, then the patient will be considered at-risk for TRALI.

The instant application is to compositions, kits and methods to determine if a person in need of a blood transfusion is at-risk for TRALI. The invention includes embodiments of methods for testing the priming activity of a blood component or serum or plasma from a patient sustaining TRALI or the priming status of neutrophils of a patient at risk for TRALI by exposing the neutrophils to samples or priming agents, and measuring the respiratory burst in response to an activating agent. The respiratory burst may then be compared to a pre-determined value to find if the patient has abnormally high respiratory burst or the plasma or serum samples have priming activity. The present invention also contemplates kits designed to measure respiratory burst, and compositions/reagents to be used in same.

What is claimed is:

1. A method of determining susceptibility to Transfusion-Related Acute Lung Injury (“TRALI”) in a patient, the method comprising:
a) removing blood from a patient;
b) isolating neutrophils from the blood;
c) exposing neutrophils to a neutrophil priming agent;
d) measuring the release of respiratory burst or its products from the neutrophils;
e) exposing neutrophils to an activating agent;
f) measuring the release of respiratory burst or its products from the neutrophils; and
g) comparing the release of steps d) and f), wherein the measured comparison between d) and f) serves as an index of susceptibility to a TRALI occurrence.

2. A method of determining susceptibility to Transfusion-Related Acute Lung Injury (“TRALI”) in patients in need of or having received blood transfusions, the method comprising:
a) removing blood from a patient;
b) isolating neutrophils from the blood;
c) exposing neutrophils to an activating agent;
d) measuring the release of respiratory burst or its products from the neutrophils; and
e) comparing the measured release of respiratory burst or its products to a benchmark measurement, wherein an increase measured release serves as an index of increased susceptibility to a TRALI occurrence.

3. The method of claim 1, wherein the respiratory burst or its products measured is at least one reactive oxygen species.

4. The method of claim 3, wherein the at least one reactive oxygen species is selected from the group consisting of: superoxide anions, hydroxyl radicals, hydrogen peroxide and singlet oxygen.

5. The method of claim 3, wherein the measuring the respiratory burst or its products is conducted by use of a cytochrome c assay.

6. The method of claim 4, wherein said cytochrome c assay comprises determining cytochrome c levels by use of electrophoresis or chromatography.

7. The method of claim 1, wherein said activating agent is selected from the group consisting of: tumor necrosis factor, fMLP, and r-phorbol-12-β-myristate-13-acetate.

8. The method of claim 1, wherein said neutrophil priming agent is selected from the group consisting of: bioactive lipids, cytokines, complement fragments, and chemokines.

9. The method of claim 1, wherein said neutrophil priming agent is selected from the group consisting of: leukotriene B₄ (LTB₄) and lysophosphatidylcholines (lyso-PCs), interleukin-18 (IL-18), granulocyte colony stimulating factor (G-CSF), donor antibodies specifically directed against recipient leukocytes, lipid soluble CD40 ligand (sCD40L), tumor necrosis factor (TNF), chemotactic peptide fMLP, 4-phorbol-12-β-myristate-13-acetate, antibodies to the HNA-3a locus, vascular endothelial growth factor (VEGF), lysophosphatidylcholines with 16-18 carbon fatty acid groups on the SN-1 position, C5a, and leukotrienes.

10. The method of claim 1, wherein said activating agent is a blood sample.

11. A kit for determining susceptibility to Transfusion-Related Acute Lung Injury (“TRALI”), the kit comprising:
at least one neutrophil priming agent;
at least one standard activating agent; and
compounds for measuring bursts and products released by neutrophils.

12. The kit of claim 11, wherein said at least one activating agent is selected from the group consisting of: tumor necrosis factor, fMLP, and r-phorbol-12-β-myristate13-acetate.

13. A kit for used for determining etiology for Transfusion-Related Acute Lung Injury (“TRALI”) reactions, testing priming state of neutrophils, or as an assay for blocking neutrophil priming states, the kit comprising:
at least on neutrophil priming agent; and
compounds for measuring products released by neutrophils.

14. The kit of claim 11, wherein said compounds for measuring bursts and products comprises antibodies to cytochrome c.

15. The kit of claim 11, wherein said compounds for measuring bursts and products comprises luminophoric reagents.

16. The kit of claim 11, said kit further comprising at least one solid support.

17. The method of claim 11, wherein said neutrophil priming agent is selected from the group consisting of: bioactive lipids, cytokines, complement fragments, and chemokines.

18. The method of claims 11, wherein said neutrophil priming agent is selected from the group consisting of: leukotriene B₄ (LTB₄) and lysophosphatidylcholines (lyso-PCs), interleukin-18 (IL-18), granulocyte colony stimulating factor (G-CSF), donor antibodies specifically directed against recipient leukocytes, lipid soluble CD40 ligand (sCD40L), tumor necrosis factor (TNF), chemotactic peptide fMLP, 4-phorbol-12-β-myristate-13-acetate, antibodies to the HNA-3a locus, vascular endothelial growth factor (VEGF), lysophosphatidylcholines with 16-18 carbon fatty acid groups on the SN-1 position, C5a, and leukotrienes.

19. A method of testing blood comprising the steps of:
a) removing blood from a patient;
b) isolating neutrophils from the blood;
c) exposing neutrophils to a neutrophil priming agent;
d) measuring the release of respiratory burst or its products from the neutrophils;
e) exposing neutrophils to an activating agent;
f) measuring the release of respiratory burst or its products from the neutrophils; and
g) comparing the release of steps d) and f), wherein the measured comparison between d) and f) serves as an index of susceptibility to a TRALI occurrence.

20. The method of claim 19, wherein the blood is obtained from a patient.

21. The method of claim 19, wherein the blood is obtained from a donor.
